(19) United States
(12) Patent Application Publication
(10) Pub. No.: US 2021/0238224 A1
(43) Pub. Date: Aug. 5, 2021

(54) METHOD OF CONSTRUCTING PROTAC BY USING DOUBLE TARGETS

(71) Applicant: THE FIRST AFFILIATED HOSPITAL OF SUN YAT-SEN UNIVERSITY, Guangzhou (CN)

(72) Inventors: Xin Wang, Guangzhou (CN); Wei Chen, Guangzhou (CN); Jiayi Yang, Guangzhou (CN); Jinjin Fan, Guangzhou (CN); Shaozhen Feng, Guangzhou (CN); Ning Luo, Guangzhou (CN)

(21) Appl. No.: 17/226,073

(22) Filed: Apr. 8, 2021

Related U.S. Application Data
(63) Continuation-in-part of application No. PCT/CN2019/104264, filed on Sep. 4, 2019.

(30) Foreign Application Priority Data
Dec. 14, 2018 (CN) 201811540463.1
Jun. 5, 2019 (CN) 201910489025.5

(51) Int. Cl. C07K 5/062; A61P 13/12

(52) U.S. Cl. CPC C07K 5/06034; A61P 13/12

(57) ABSTRACT

The present invention provides a method for constructing PROTAC by using double targets, and a specific construction method and application of a double-target. PROTAC constructed by using the method. The present invention proposes for the first time the concept of PROTAC with double-target design, namely, the Target protein I is degraded by PROTAC when a specific E3 is selected, meanwhile, the Target protein II is increased for the degradation of the E3 natural substrate protein is hindered due to the competition of PROTAC to E3. By using this method, the present invention constructs for the first time a double-target PROTAC which is capable of not only degrading Smad3 via target ubiquitination but also simultaneously up-regulating the HIF-α protein level, which theoretically plays the role of renal protection, such as anti-fibrosis and the treatment of renal anemia, via multi channels.

### CROSS-REFERENCE TO RELATED APPLICATIONS

The present application is a Continuation-In-Part Application of PCT application No. PCT/CN2019/104264 filed on Sep. 4, 2019, which claims the benefit of Chinese Patent Applications No. 201910489025.5 filed on Jun. 5, 2019 and No. 201811540463.1 filed on Dec. 14, 2018, the contents of which are hereby incorporated by reference.

### TECHNICAL FIELD

The present invention relates to a method of constructing PROTAC by using double targets.

### BACKGROUND ART

Following the changes of lifestyles, the underlying disease incidence, such as diabetes, hypertension and other Chronic Kidney Diseases (CKD), increases day by day, and CKD has become a worldwide public health problem. Currently, effective clinical treatments are very limited for CKD patients, and quite a few CKD patients eventually progressed into End Stage Renal Disease (ESRD), which requires expensive kidney replacement therapy and brings a great burden to family and society. Therefore, it is currently a priority research subject in our “National Medical Development Strategy” as for how to prevent CKD from progressing into to ESRD.

It has been made clear that glomerulosclerosis and renal interstitial fibrosis are pathological bases of CKD progression, and Smad3 inhibitor, miR-21 antagonist and other methods have been applied to successfully block the process of renal fibrosis in animal models, but there is still no drug that can be successfully applied to clinical practice. An important reason for the failure of these anti-fibrosis target drugs is that both the inhibitor and the antagonist lack precise targeting but have large side effects. In recent years, by using the function characteristics of the ubiquitin proteasome pathway to specifically degrade protein substrate, people have constructed Proteolysis Targeting Chimera (PROTAC), through which target protein is recruited to E3 ligase for ubiquitination and then degraded via the proteasome pathway, see FIG. 1.

PROTAC has extremely high targeting; moreover, it has an action mechanism similar to catalytic reaction, so it is capable of obtaining extremely high activity by using drugs of a very small dose, rather than of an equal molar amount. As a compound, PROTAC does not have immunogenicity; it is simple in structure, can be easily synthesized, and can easily and flexibly travel within the bodies and enter cells. As a result, it is practically valuable from the perspective of clinical application. In the past two years, at least two PROTAC companies have obtained large-scale financing and huge cooperation agreements with Merck, Roche and other companies. At present, there has been many preclinical experiments using PROTAC, but they are all aimed at various tumor targets, and the application of PROTAC in kidney field is basically blank.

Previously, we combined the computer virtual screening technology and the Surface Plasmon Resonance (SPR) technology for the first time, and have successfully screened out small molecules specifically bind to the target protein Smad3 from the small molecule library, and have successfully constructed PROTAC capable of degrading intracellular Smad3 by means of targeting ubiquitination with such small molecules serving as the target protein recognition ligand and the pentapeptide structure of the hypoxia-inducible factor (HIF) as E3 recognition ligand. However, when polypeptide is used as the E3 recognition ligand, the molecules of the entire PROTAC are too large, so the cell permeability is bad, and a higher concentration is required for entering into the cells. In order to solve the problem of poor cell permeability of PROTAC, Crews CM used the small molecule pomalidomide as the E3 recognition ligand of PROTAC for the first time in 2015, and significantly improved the cell permeability of PROTAC by making its effective concentration to be as low as nM.

When small molecules are used as the E3 recognition ligand of PROTAC, the membrane permeability of PROTAC can be greatly enhanced; however, due to the high affinity of small molecules to E3, the ubiquitination and degradation of E3 natural substrate protein may be hindered, which causes accumulation of E3 natural substrate protein and produces off-target effects.

### SUMMARY OF THE INVENTION

In order to prevent the possible off-target effects of PROTAC, and based on the understanding of the E3 ligase action mechanism in the formation process of ubiquitin chains, the present invention proposes construction of PROTAC by using double targets for the first time: the target protein recognition ligand determines what kind of target protein is to be degraded by PROTAC (Target I), and the E3 recognition ligand selects a specific E3 so as further to determine what kind of E3 ligase natural substrate protein will be increased by PROTAC (Target II). By reasonable selection of two targets, the off-target effects be effectively avoided, and the synergistic effect can also be achieved, as a result, the effects of PROTAC will be secure and maximized. The present invention also provides a construction method and application of the double-target PROTAC.

In order to realize the above object, the following technical solution is adopted: a method of constructing PROTAC by using double targets, including steps of:

(1) using harmful intracellular protein as a first target protein, and screening a small molecular compound specifically bind to the first target protein to serve as a recognition ligand of the first target protein;

(2) selecting a protective protein within cells naturally expressing the first target protein to serve as a second target protein, using a specific E3 ligase of the second target protein as E3 of PROTAC recognition, and screening and determining a small molecular compound capable of specifically binding to E3 ubiquitin ligase to serve as a recognition ligand of the E3 ubiquitin ligase;

(3) screening a compound capable of stably binding to both the recognition ligand of the first target protein obtained in Step (1) and the recognition ligand of E3 ubiquitin ligase obtained in Step (2) to serve as Linker; and

(4) connecting the recognition ligand of the first target protein obtained in Step (1) and the recognition ligand of E3 ubiquitin ligase obtained in Step (2) by using the Linker obtained in Step (3) so as to obtain the double-target PROTAC.

Preferably, the first target protein is Smad3, and the recognition ligand of the first target protein is a compound expressed by Formula (I),

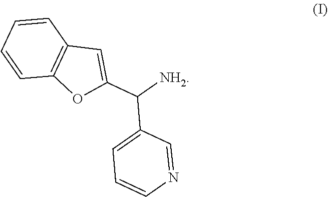 [SMILES: NC(c1cccnc1)c1cc2ccccc2o1]

Preferably, the second target protein is HIF-α protein.

Preferably, the E3 ubiquitin ligase is VHL E3 ubiquitin ligase.

Preferably, the recognition ligand of the E3 ubiquitin ligase is a compound expressed by Formula (H),

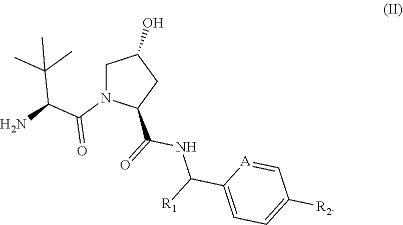

Preferably, the Linker is butanedioic acid.

Preferably, the double-target PROTAC is a compound expressed by Formula (III),

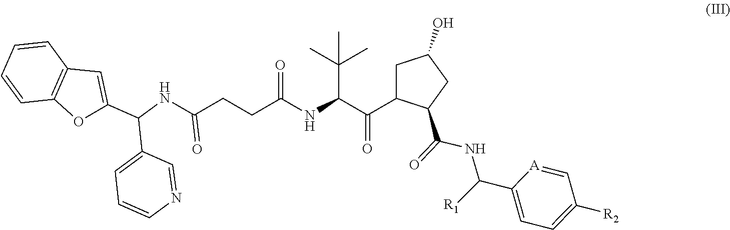

wherein: R₁ is hydrogen, straight-chain alkyl or substituted straight-chain alkyl, cycloalkyl, alkenyl or substituted alkenyl, carbonyl or substituted carbonyl, five-membered or six-membered heterocycle, benzene ring or benzene ring containing substituent; R₂ is hydrogen, straight-chain alkyl or substituted straight-chain alkyl, cycloalkyl, carbonyl or substituted carbonyl, five-membered or six-membered heterocycle, five-membered aromatic heterocycle or five-membered aromatic heterocycle containing substituent, benzene ring or benzene ring containing substituent, six-membered aromatic heterocycle or six-membered aromatic heterocycle containing substituent; and A is carbon atom or nitrogen atom.

Preferably, R₁ is hydrogen, straight-chain alkyl or substituted straight-chain alkyl; R₂ is hydrogen, straight-chain alkyl or substituted straight-chain alkyl, cycloalkyl, five-membered or six-membered heterocycle, five-membered aromatic heterocycle or five-membered aromatic heterocycle containing substituent, benzene ring or benzene ring containing substituent, six-membered aromatic heterocycle or six-membered aromatic heterocycle containing substituent; and A is carbon atom or nitrogen atom.

Preferably, R₁ is hydrogen, methyl, ethyl, isopropyl; and R₂ is hydrogen, methyl, ethyl, isopropyl, morpholine, piperazine, thiophene or thiophene ring containing substituent, thiazole or thiazole ring containing substituent, benzene ring or benzene ring containing substituent, pyridine ring or pyridine ring containing substituent. A is carbon atom or nitrogen atom.

Preferably, the structural formula of the PROTAC is one of the structural formulae below:

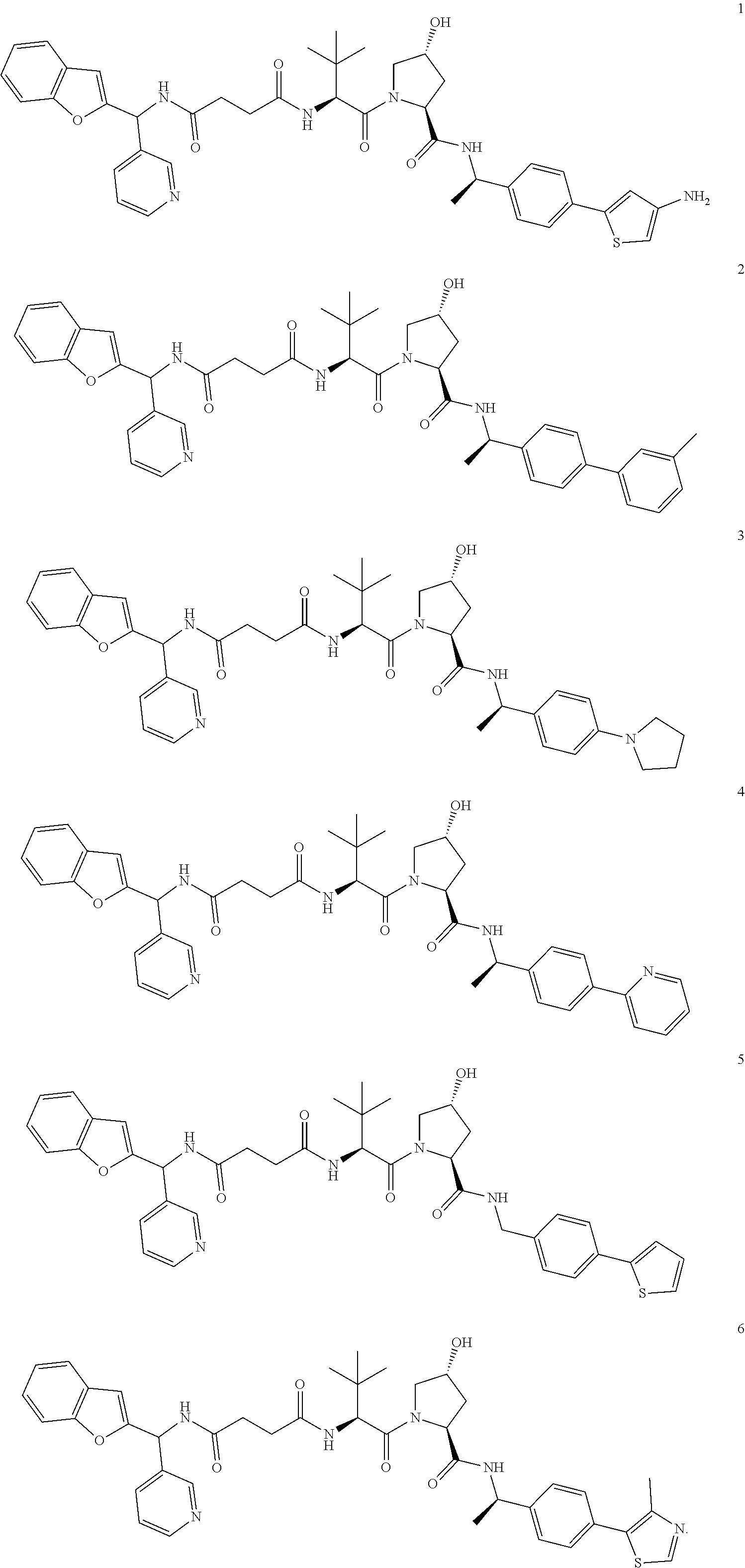

Furthermore, the drug includes a pharmaceutically acceptable salt, carrier and/or excipient.

The present invention provides a double-target PROTAC constructed by using the above-mentioned method.

The present invention provides the use of the afore-mentioned double-target PROTAC in the preparation of drugs for anti-fibrosis, treatment of renal anemia, diabetes, diabetic nephropathy and/or protection of renal function.

Preferably, the fibrosis is renal fibrosis.

The beneficial effects of the present invention are as below:

The present invention provides a method for constructing PROTAC by using double targets, and a specific construction method and application of a double-target. PROTAC constructed by using the method. The present invention proposes for the first time the concept of PROTAC with double-target design, namely, the Target protein I is degraded by PROTAC when a specific E3 is selected, meanwhile, the Target protein II is increased for the degradation of the E3 natural substrate protein is hindered due to the competition of PROTAC to E3. By using this method, the present invention constructs for the first time a double-target PROTAC which is capable of not only degrading Smad3 via target ubiquitination but also simultaneously up-regulating the HIF-α protein level, which theoretically plays the role of renal protection, such as anti-fibrosis and the treatment of renal anemia, via multi channels.

### DESCRIPTION OF THE DRAWINGS

FIG. 1 is a diagram of target protein degradation mediated by PROTAC.

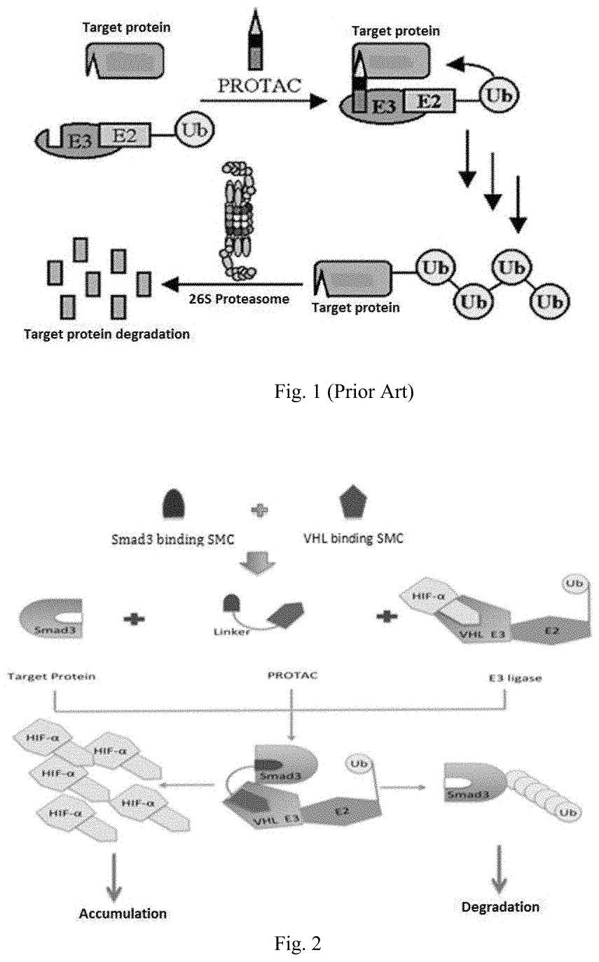

FIG. 2 is a mode pattern of Smad3/HIF-α double-target PROTAC construction mechanism. Note: SMC: small molecular compound.

FIG. 3 is a mode pattern of research hypothesis.

FIG. 4 is a combination mode pattern of Smad3 target protein ligand and Smad3 protein in Example 1 of the present invention.

FIGS. 5(A), 5(B), 5(C) and 5(D) are diagrams showing the structure identification (MS and HPLC) of the double-target PROTAC in Example 1 of the present, invention. Note: FIGS. 5(A) and 5(B) are respectively MS diagrams of butanedioic acid and PEG; FIGS. 5(C) and 5(D) are respectively HPLC diagrams of butanedioic acid and PEG.

FIGS. 6(A) and 6(B) are diagrams showing the activity comparison of double-target PROTAC with different Linkers in Example 1 of the present invention (Western blot). Note: comparison with TGF-β1 control group, *P<0.05, **P<0.01.

FIG. 7 is a diagram showing the pharmacological effects of Smad3/HIF-α double-target PROTAC on renal fibroblasts in Example 1 of the present invention.

### EMBODIMENTS

In order to better explain the purpose, the technical solution and the advantages of the present invention, the present invention will be further elaborated with reference to the specific example.

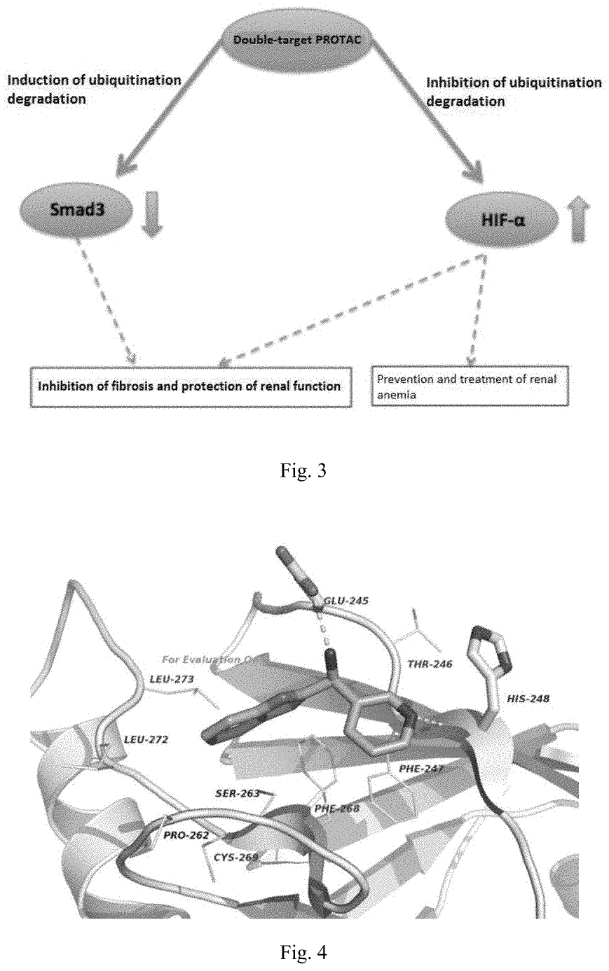

Based on our research basis for constructing a single-target PROTAC, the present invention proposes for the first time to construct a Smad3/HIF-α double-target. PROTAC, and the construction mechanism is shown in FIG. 2.

In our anticipation, the combination of the different anti-fibrosis mechanism effects of two targets can not only significantly enhance the renal protection of PROTAC, but also complement with each other, i.e. to overcome, by means of Smad3 degradation, the pro-fibrosis effect that HIF-1α may have in the early stage of renal injury while preserving the powerful renal protection of HIF-2α.

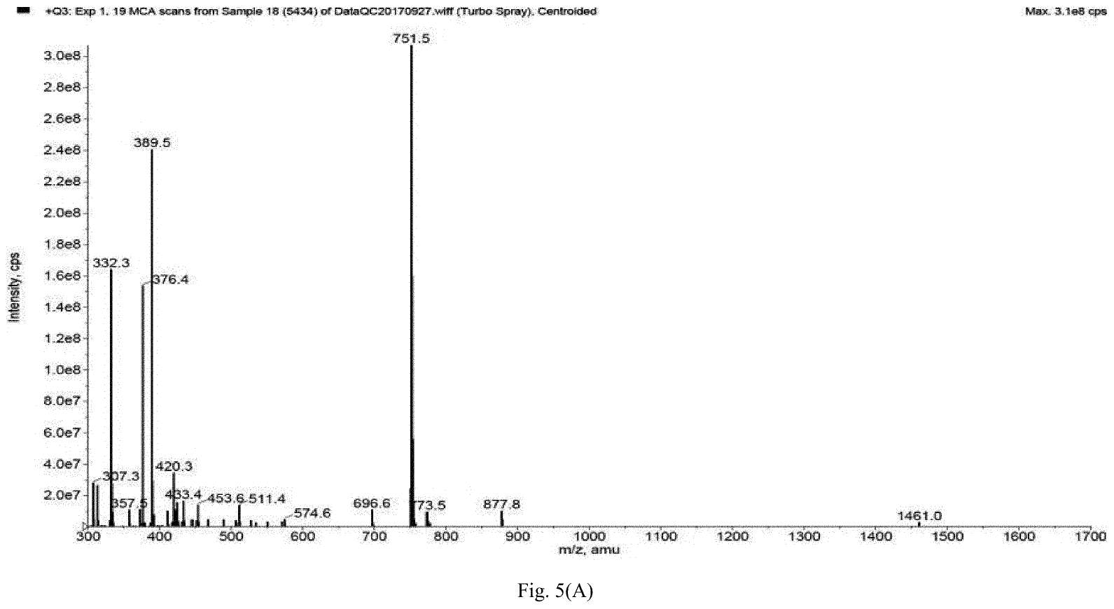

In summary, we put forward the following research hypothesis: in the progress of CKD, due to the excessive activation of Smad3 signal and relative insufficiency of HIF-α, the application of Smad3/HIF-α double-target PROTAC can block the fibrosis of the Smad3 signal pathway, as well as has the function of HIF-α stabilization, thereby achieving multiple goals of double targets, inhibiting renal fibrosis, renal function protection, and renal anemia prevention and treatment through multiple pathways, see FIG. 3.

Based on our preliminary research basis of screening specifically recognized Smad3 protein small molecules as the target protein recognition ligand, small molecules specifically bind to VHL are used as the E3 recognition ligand to construct PROTAC, and Smad3/HIF-α double-target PROTAC is constructed and synthesized, which can significantly degrade intercellular Smad3 protein of rat kidney fibroblasts while up-regulating the protein level of HIF-2a.

### Example 1

I. Seek Small Molecules Specifically Bind to Target Protein Smad3 by Combining Computer Virtual Screening Technology and SPR Technology

Based on our previous research results, we have successfully screened out a small molecule that can specifically bind to the target protein Smad3, the structural formula is shown in Formula (I), and it is confirmed that PROTAC constructed by using this small molecule as the target protein ligand can degrade Smad3 protein via target ubiquitination, as a result, for the double-target PROTAC newly constructed this time, we still use this small molecule as the target protein ligand.

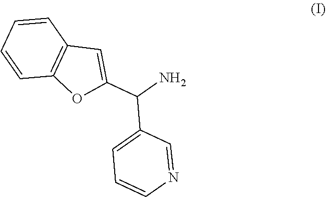 [SMILES: NC(c1cccnc1)c1cc2ccccc2o1]

II. Determination of the Binding Site of Target Protein Small Molecule Ligand with the Linker

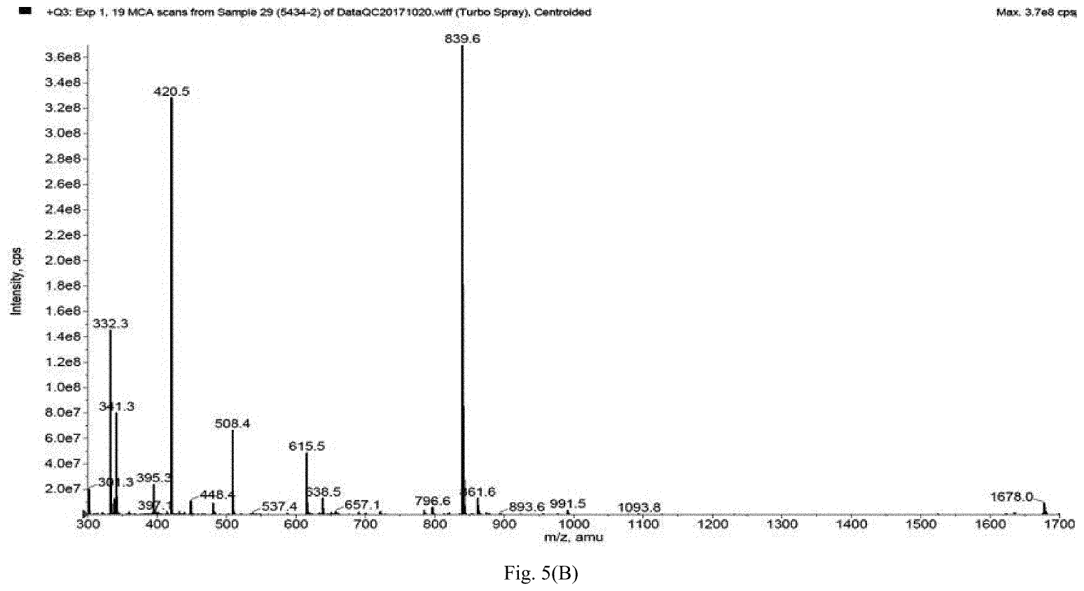

Molecule docking is performed for the previously screened small molecules and Smad3, see FIG. 4. It can be seen that the amine group (—NH₂) on the upper part of No. 8 small molecule forms a hydrogen bond with the carboxyl group at the end of Glu-245 residue of Smad3, N on the pyridine ring of the molecule forms a hydrogen bond with —NH on the main chain of the residue His-248, and the dihydrogen bond holds the small molecule here; secondly, the pyridine ring on the right part of the small molecule forms a π-π interaction with the benzene ring at the end of the Phe-247 residue, and the benzofuran ring on the left part and the benzene ring at the end of the Phe-268 residue also forms a π-π interaction. From the interaction diagram, it can be seen that the benzofuran ring and pyridine ring of the small molecule extend into the interior of the protein and adapt to the active pocket of the protein, while the amino group at the upper part faces the exterior space of the protein, so the modification of the amino group has little effect on the binding activity of No. 8 small molecule and the Smad3 protein, and can serve as a site connecting with Linker. In addition, the amino group is an active group for chemical reaction, and its link with Linker can be easily realized.

III. Determination of the Binding Site of VHL E3 Recognition Ligand Small Molecule with the Linker

Small molecule specifically bind to VHL is adopted in this invention as VHL E3 recognition ligand, and the structural formula is shown by Formula (II),

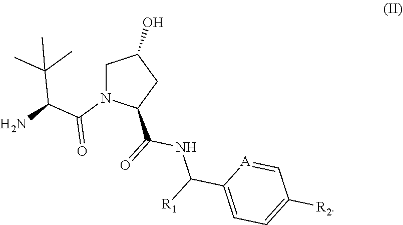

IV. Construction of Double-Target PROTAC with Different Linkers

Under the premise of non-toxicity, good water solubility and small molecular weight, the present invention initially selects two small molecules, including a long one (polyethylene glycol, PEG) and a short one (butanedioic acid) as Linkers for constructing double-target PROTAC: PROTAC-PEG (see Compound 7) and PROTAC-butanedioic acid (see Compound 1).

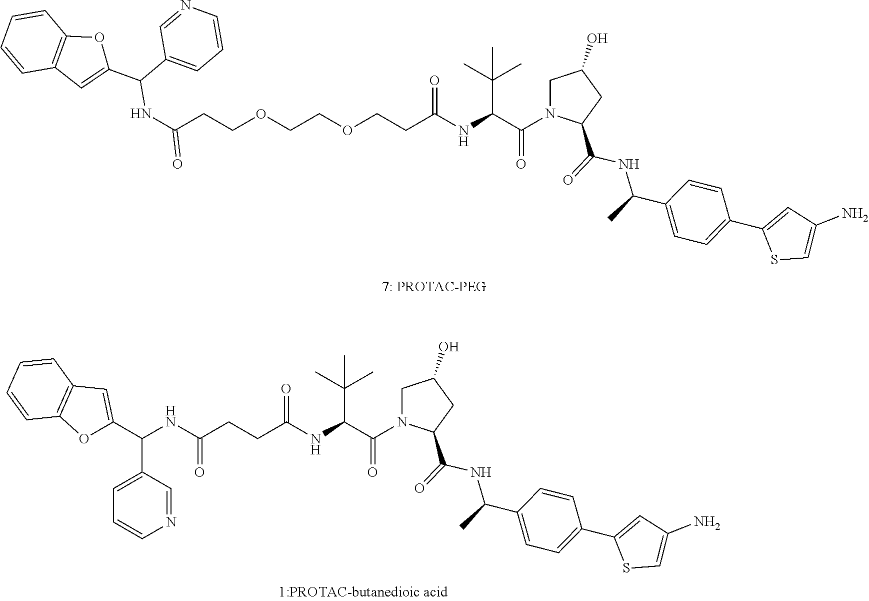 [SMILES: C[C@@H](NC(=O)[C@@H]1C[C@@H](O)CN1C(=O)[C@@H](NC(=O)CCOCCOCCC(=O)NC(c1cccnc1)c1cc2ccccc2o1)C(C)(C)C)c1ccc(-c2cc(N)cs2)cc1 ; C[C@@H](NC(=O)[C@@H]1C[C@@H](O)CN1C(=O)[C@@H](NC(=O)CCC(=O)NC(c1cccnc1)c1cc2ccccc2o1)C(C)(C)C)c1ccc(-c2cc(N)cs2)cc1]

V. Structural Verification of PROTAC with Different. Linkers

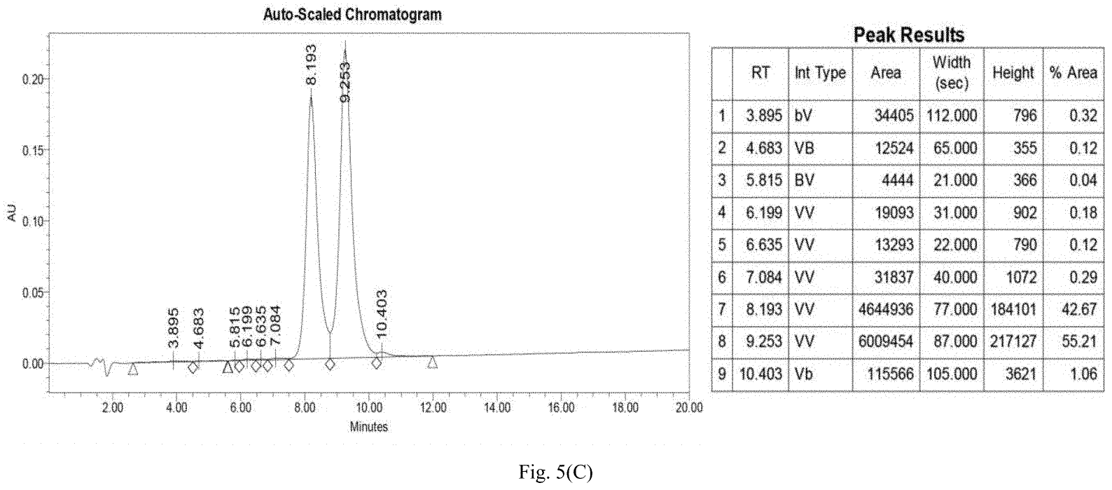

The structure of PROTAC is analyzed to be correct by means of mass spectrometry (MS) and high performance liquid chromatography (HPLC), see FIGS. 5(A), 5(B), 5(C) and 5(D).

VI. Activity Comparison of Double-Target PROTAC Constructed by Different Linkers

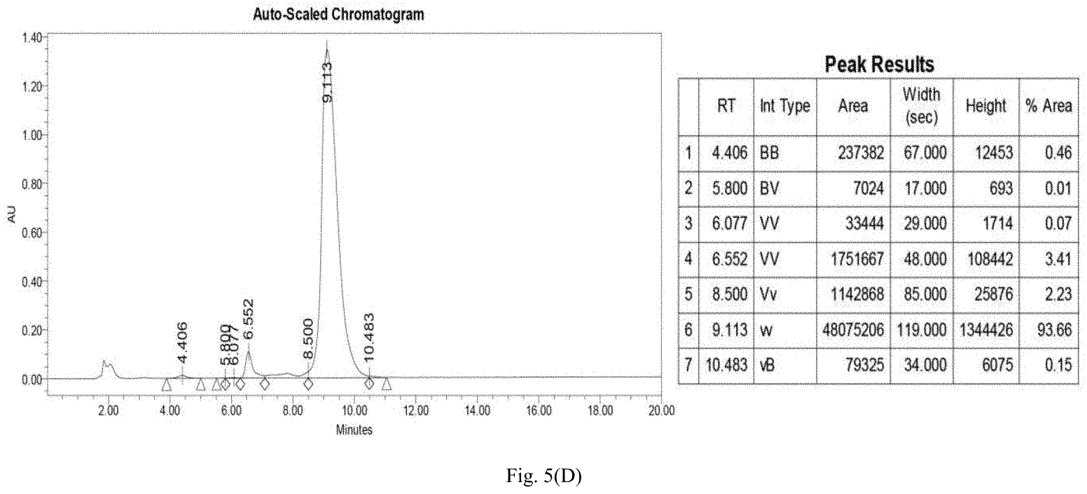

Rat kidney fibroblasts (NRK49F cell line) are cultured in vitro, passage 1:3 or 1:4 to six-well plates before cell fusion, and are divided into a TGF-β1 control group (TGF-β1 10 ng/ml), a PROTAC-PEG action group (TGF-β1 10 ng/ml+PROTAC-PEG of different concentrations: 1, 5, 25, 125 nM), and a PROTAC-butanedioic acid action group (TGF-β1 10 ng/ml+PROTAC-butanedioic acid of different concentrations: 1, 5, 25, 125 nM); the cells are lysed after 48 hours of culture and are used for Western blot. The results show that PROTAC-butanedioic acid degrades the target protein Smad3 in a concentration-dependent manner, its activity is significantly higher than that of PROTAC-PEG, and its effective concentration is as low as 25 nM, see FIGS. 6(A) and 6(B).

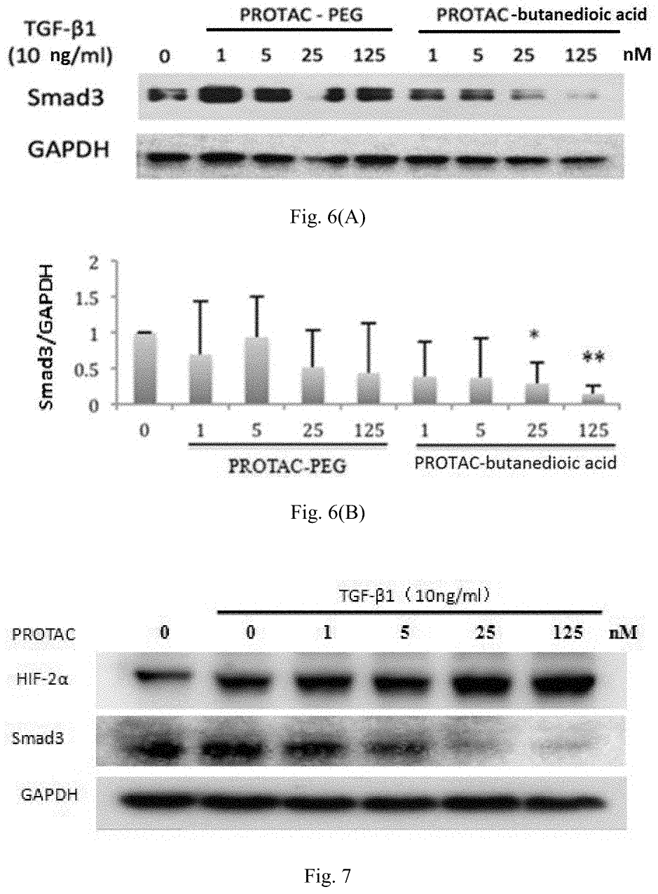

By means of experiments in vitro, it has been confirmed that the PROTAC can degrade intracellular Smad3 in a concentration-dependent manner, and at the same time significantly up-regulate the protein level of HIF-2a, and the effective concentration is lower than 25 nM, see FIG. 7.

Finally, it should be noted that the above embodiments are only used to illustrate the technical solutions of the present invention, rather than limiting the protection scope of the present invention. Although the present invention has been described in detail with reference to preferable example, ordinary technicians in this field should understand that the technical solution of the present invention can be modified or equivalently replaced without departing from the substance and scope of the technical solution of the present invention.

What s claimed is:

1. A method of constructing PROTAC by using double targets, wherein including steps of:
(1) using harmful intracellular protein as a first target protein, and screening a small molecular compound specifically bind to the first target protein to serve as a recognition ligand of the first target protein;
(2) selecting a protective protein within cells naturally expressing the first target protein to serve as a second target protein, using a specific E3 ligase of the second target protein as E3 of PROTAC recognition, and screening and determining a small molecular compound capable of specifically binding to E3 ubiquitin ligase to serve as a recognition ligand of the E3 ubiquitin ligase;
(3) screening a compound capable of stably binding to both the recognition ligand of the first target protein obtained in Step (1) and the recognition ligand of E3 ubiquitin ligase obtained in Step (2) to serve as Linker; and
(4) connecting the recognition ligand of the first target protein obtained in Step (1) and the recognition ligand of E3 ubiquitin ligase obtained in Step (2) by using the Linker obtained in Step (3) so as to obtain the double-target PROTAC.

2. The method according to claim 1, wherein the first target protein is Smad3, and the recognition ligand of the first target protein is a compound expressed by Formula (I),

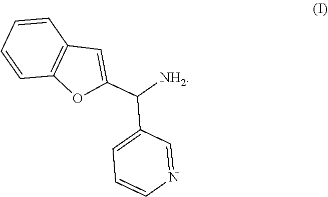 [SMILES: NC(c1cccnc1)c1cc2ccccc2o1]

3. The method according to claim 1, wherein the second target protein is HIF-α protein.

4. The method according to claim 3, wherein the E3 ubiquitin ligase is VHL E3 ubiquitin ligase.

5. The method according to claim 1, wherein the recognition ligand of the E3 ubiquitin ligase is a compound expressed by Formula (II),

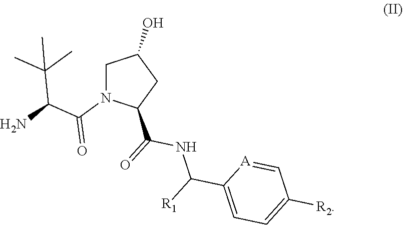

6. The method according to claim 1, wherein the Linker is butanedioic acid.

7. The method according to claim 1, wherein the double-target PROTAC is a compound expressed by Formula (III), 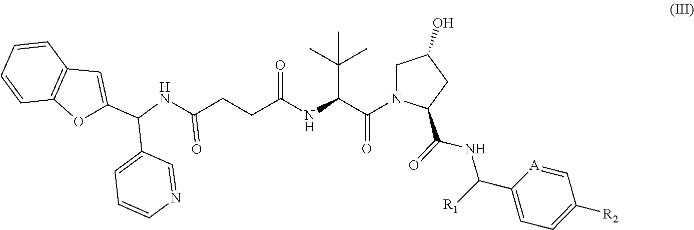
wherein: R₁ is hydrogen, straight-chain alkyl or substituted straight-chain alkyl, cycloalkyl, alkenyl or substituted alkenyl, carbonyl or substituted carbonyl, five-membered or six-membered heterocycle, benzene ring or benzene ring containing substituent; R₂ is hydrogen, straight-chain alkyl or substituted straight-chain alkyl, cycloalkyl, carbonyl or substituted carbonyl, five-membered or six-membered heterocycle, five-membered aromatic heterocycle or five-membered aromatic heterocycle containing substituent, benzene ring or benzene ring containing substituent, six-membered aromatic heterocycle or six-membered aromatic heterocycle containing substituent; and A is carbon atom or nitrogen atom.

8. The method according to claim 7, wherein R₁ is hydrogen, straight-chain alkyl or substituted straight-chain alkyl; R₂ is hydrogen, straight-chain alkyl or substituted straight-chain alkyl, cycloalkyl, five-membered or six-membered heterocycle, five-membered aromatic heterocycle or five-membered aromatic heterocycle containing substituent, benzene ring or benzene ring containing substituent, six-membered aromatic heterocycle or six-membered aromatic heterocycle containing substituent; and A is carbon atom or nitrogen atom.

9. The method according to claim 8, wherein R₁ is hydrogen, methyl, ethyl, isopropyl; R₂ is hydrogen, methyl, ethyl, isopropyl, morpholine, piperazine, thiophene or thiophene ring containing substituent, thiazole or thiazole ring containing substituent, benzene ring or benzene ring containing substituent, pyridine ring or pyridine ring containing substituent; and A is carbon atom or nitrogen atom.

10. The method according to claim 9, wherein the structural formula of the PROTAC is one of the structural formulae below:

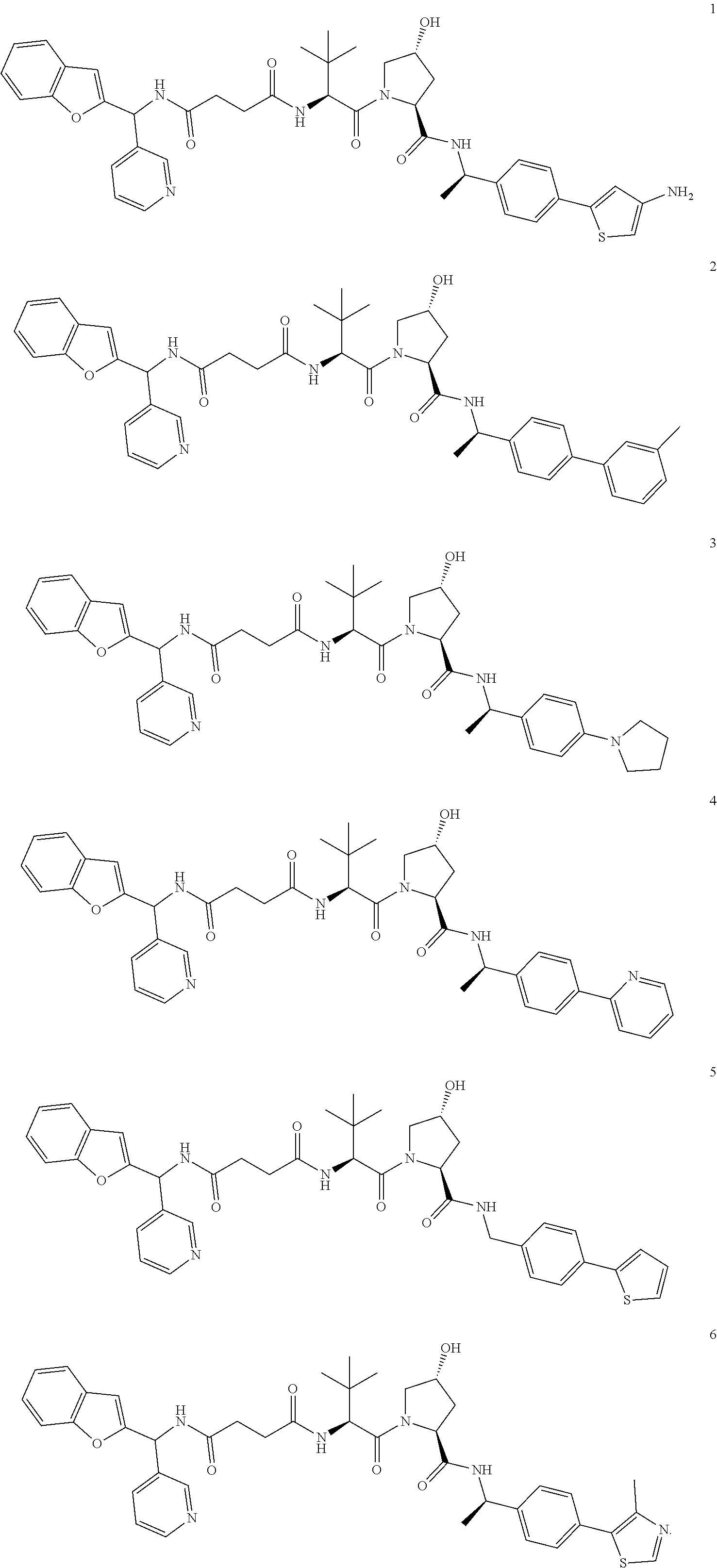

11. A double-target PROTAC constructed by using the method according to claim 1.

12. A drug for anti-fibrosis, treatment of renal anemia, diabetes, diabetic nephropathy and/or protection of renal function comprising the double-target PROTAC according to claim 11.

13. The drug according to claim 2, wherein the fibrosis is renal fibrosis.
